(19) United States
(12) Patent Application Publication
(10) Pub. No.: US 2023/0333077 A1
(43) Pub. Date: Oct. 19, 2023

(54) COMPOSITION ANALYSIS DEVICE AND COMPOSITION ANALYSIS METHOD FOR FUEL GAS, PRIME MOVER CONTROL DEVICE INCLUDING COMPOSITION ANALYSIS DEVICE, AND PRIME MOVER CONTROL METHOD INCLUDING COMPOSITION ANALYSIS METHOD

(71) Applicant: MITSUBISHI HEAVY INDUSTRIES, LTD., Tokyo (JP)

(72) Inventors: Toshifumi SASAO, Kanagawa (JP); Keita NAITO, Kanagawa (JP); Akinori HAYASHI, Kanagawa (JP)

(21) Appl. No.: 18/027,451

(22) PCT Filed: Oct. 25, 2021
(86) PCT No.: PCT/JP2021/039257

§ 371 Date: Mar. 21, 2023

(30) Foreign Application Priority Data
Oct. 29, 2020 (JP) 2020-181893

(51) Int. Cl. G01N 33/22; G01N 9/36; G01N 25/36; F23N 5/26; F02C 9/26

(52) U.S. Cl. CPC G01N 33/225; G01N 9/36; G01N 25/36; F23N 5/265; F02C 9/26; F23N 2241/20

(57) ABSTRACT

A composition analysis device for fuel gas containing inert gas and flammable gas includes: a heating value measurement device for measuring a heating value per unit amount of the fuel gas; a density measurement device for measuring a density of the fuel gas; and a control device including a composition calculation unit for calculating a composition of the fuel gas using the heating value measured by the heating value measurement device and the density measured by the density measurement device.

### TECHNICAL FIELD

The present disclosure relates to a composition analysis device and a composition analysis method for fuel gas, a prime mover control device including the composition analysis device, and a prime mover control method including the composition analysis method.

The present application claims priority based on Japanese Patent Application No. 2020-181893 filed on Oct. 29, 2020, the entire content of which is incorporated herein by reference.

### BACKGROUND ART

When fuel gas supplied to a gas turbine contains inert gas such as nitrogen, the concentration of the inert gas in the fuel gas affects the combustibility of the fuel gas. Patent Document 1 describes a fuel flow control device capable of stably burning fuel gas in a gas turbine even when using fuel gas whose concentration of inert gas changes over time. This fuel flow control device measures the concentration of inert gas in the fuel gas and controls the supply flow rate of the fuel gas based on the measured inert gas concentration.

### CITATION LIST

### Patent Literature

Patent Document 1: JP2005-127197A

### SUMMARY

### Problems to be Solved

However, gas chromatography is generally used to measure the concentration of inert gas in fuel gas, but gas chromatography requires a long detection time, so if the concentration of inert gas in the fuel gas changes from moment to moment, the fuel flow control device described in Patent Document 1 makes it difficult to control the fuel flow rate.

In view of the above circumstances, an object of at least one embodiment of the present disclosure is to provide a composition analysis device and a composition analysis method for fuel gas, a prime mover control device including the composition analysis device, and a prime mover control method including the composition analysis method whereby it is possible to analyze the composition of fuel gas quickly.

### Solution to the Problems

To achieve the above object, a composition analysis device for fuel gas according to the present disclosure is a composition analysis device for fuel gas containing inert gas and flammable gas, comprising: a heating value measurement device for measuring a heating value per unit amount of the fuel gas; a density measurement device for measuring a density of the fuel gas; and a control device including a composition calculation unit for calculating a composition of the fuel gas using the heating value measured by the heating value measurement device and the density measured by the density measurement device.

Further, a composition analysis method for fuel gas according to the present disclosure is a composition analysis method for fuel gas containing inert gas and flammable gas, comprising: a step of measuring a heating value per unit amount of the fuel gas; a step of measuring a density of the fuel gas; and a step of calculating a composition of the fuel gas using the heating value and the density measured.

### Advantageous Effects

With the composition analysis device and the composition analysis method for fuel gas according to the present disclosure, the heating value per unit amount of the fuel gas and the density of the fuel gas, which can be quickly measured, are measured, and these measured values are used to analyze the composition of the fuel gas, so it is possible to analyze the composition of the fuel gas including inert gas and flammable gas quickly.

### BRIEF DESCRIPTION OF DRAWINGS

FIG. 1 is a configuration diagram of a composition analysis device for fuel gas and a prime mover control device including the composition analysis device according to an embodiment of the present disclosure.

FIG. 2 is a schematic diagram showing the configuration of a combustor of a gas turbine provided with the composition analysis device for fuel gas according to an embodiment of the present disclosure.

FIG. 3 is a schematic configuration diagram of a control device of the composition analysis device for fuel gas according to an embodiment of the present disclosure.

FIG. 4 is a graph for describing a principle of calculating the composition of fuel gas using the density and heating value of the fuel gas by the composition analysis device for fuel gas according to an embodiment of the present disclosure.

FIG. 5 is a graph for describing another principle of calculating the composition of fuel gas using the density and heating value of the fuel gas by the composition analysis device for fuel gas according to an embodiment of the present disclosure.

FIG. 6 is a diagram showing an example of a control flow for calculating a fuel ratio from the concentration of inert gas in the fuel gas and the heating value of the fuel gas by the prime mover control device according to an embodiment of the present disclosure.

### DETAILED DESCRIPTION

Hereinafter, a composition analysis device and a composition analysis method for fuel gas according to embodiments of the present disclosure will be described with reference to the drawings. The following embodiments are illustrative and not intended to limit the present disclosure, and various modifications are possible within the scope of technical ideas of the present disclosure.

### Configuration of Composition Analysis Device for Fuel Gas and Prime Mover Control Device According to Embodiment of Present Disclosure

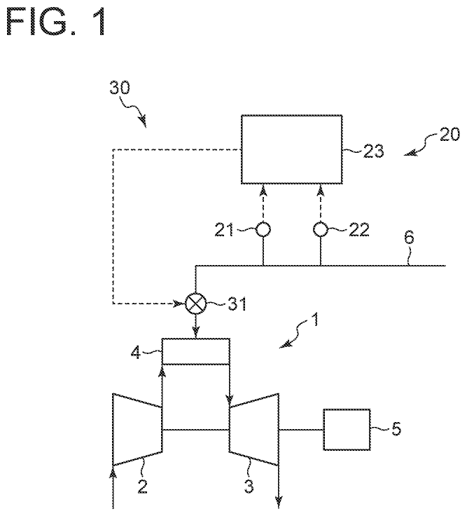

As shown in FIG. 1, a composition analysis device 20 for fuel gas according to an embodiment of the present disclosure is to analyze the composition of fuel gas supplied to a gas turbine 1 which is a prime mover. Fuel gas contains fuel components, namely, flammable gas such as hydrocarbon fuel and inert gas such as nitrogen, and the composition of fuel gas analyzed by the composition analysis device 20 specifically means the concentration of inert gas or the concentration of flammable gas or both in the fuel gas. In the embodiments described below, the analysis of the composition of fuel gas is described with the embodiment where the concentration of inert gas in the fuel gas is determined, which is essentially synonymous with determining the concentration of flammable gas in the fuel gas or the concentration of each of inert gas and flammable gas.

The gas turbine 1 includes a compressor 2 for generating compressed air, a combustor 4 for generating combustion gas from the compressed air and fuel gas, and a turbine 3 configured to be rotationally driven by the combustion gas. The turbine 3 is connected to a generator 5 driven by the turbine 3. A fuel supply line 6 connected at one end to a fuel supply source (not shown) is connected at the other end to the combustor 4.

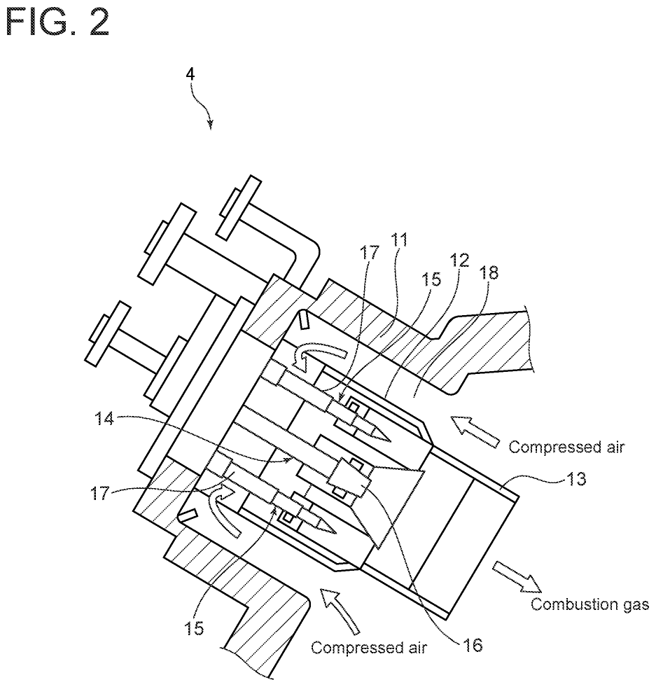

As shown in FIG. 2, the combustor 4 includes a combustor casing 11 and a combustor basket 12 disposed in the combustor casing 11 with predetermined spacing in the radial direction with respect to the axis of the combustor casing 11. A transition piece 13 is connected to a tip portion of the combustor basket 12. Between the combustor casing 11 and the combustor basket 12, a ring-shaped passage 18 is formed through which the compressed air compressed by the compressor 2 (see FIG. 1) flows. In the combustor basket 12, a pilot combustion burner 14, which is a first burner, and a plurality of main combustion burners 15, which are second burners, disposed so as to surround the pilot combustion burner 14 are arranged. The pilot combustion burner 14 includes a pilot nozzle 16, which is a first nozzle, and each of the main combustion burners 15 includes a main nozzle 17, which is a second nozzle.

As shown in FIG. 1, the composition analysis device 20 includes a density measurement device 21 and a heating value measurement device 22, disposed on the fuel supply line 6, for measuring the density of fuel gas and the heating value per unit amount (unit volume or unit mass) of fuel gas, respectively, and a control device 23 electrically connected to the density measurement device 21 and the heating value measurement device 22. The control device 23 inputs measured values of the density ρ₀ and the heating value LHV₀ as electric signals from the density measurement device 21 and the heating value measurement device 22, respectively. The density measurement device 21 and the heating value measurement device 22 are not limited to particular configurations, but may have any configuration as long as they can measure the density and the heating value. The density measurement device 21 and the heating value measurement device 22 may be separate devices, or may be one device capable of measuring both the density and the heating value. As an example of the latter configuration, for example, an explosion-proof calorimeter can be used. The control device 23 includes a central processing unit (CPU), a random access memory (RAM), a read only memory (ROM), a hard disk drive (HDD), and a control circuit, not depicted, and is realized by executing a predetermined control program stored in the ROM by the CPU.

The gas turbine 1 may be equipped with a prime mover control device 30 for controlling the operation of the gas turbine 1 based on the composition of fuel gas analyzed by the composition analysis device 20. The prime mover control device 30 is provided with the composition analysis device 20. In the embodiments described below, the control of the operation of the gas turbine 1 is described using the example of adjusting a fuel ratio, which is the ratio of fuel gas supplied to each of the pilot nozzle 16 (see FIG. 2) and the main nozzles 17 (see FIG. 2), but is not necessarily limited to this embodiment. For example, as the control of the operation of the gas turbine 1, in order to eliminate poor combustion due to inert gas, multiple combustion burners may be switched and controlled to achieve a higher fuel-air ratio. In this embodiment, a fuel ratio control unit 31 (e.g., a control valve for controlling the flow rate of fuel gas supplied to each of the pilot nozzle 16 and the main nozzles 17) is provided downstream of the density measurement device 21 and the heating value measurement device 22 on the fuel supply line 6 as one of the constituent elements of the prime mover control device 30. In this embodiment, the fuel ratio control unit 31 is provided outside the control device 23 as the control valve, for example, but the ratio of fuel gas can also be adjusted by program control. In this case, a fuel ratio control unit 31 configured to control the fuel ratio by a program may be provided inside the control device 23, for example. In this case, since the fuel ratio control unit 31 is provided inside the control device 23, the number of components of the prime mover control device 30 can be reduced.

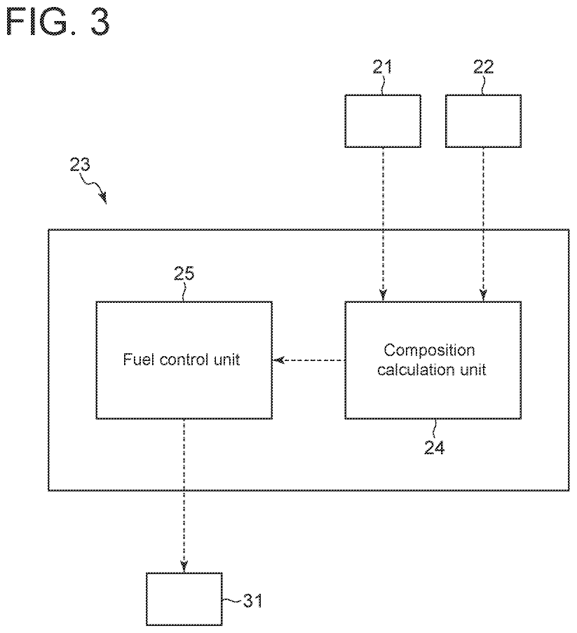

As shown in FIG. 3, the control device 23 as a constituent element of the composition analysis device 20 includes a composition calculation unit 24 for calculating the composition of the fuel gas using the density measured by the density measurement device 21 and the heating value measured by the heating value measurement device 22. Further, when the prime mover control device 30 provided with the composition analysis device 20 is installed in the gas turbine 1, the control device 23 includes a fuel control unit 25 for calculating a fuel control command to correct the fuel ratio and outputting the command to the fuel ratio control unit 31.

Outside the control device 23, the above-described fuel ratio control unit 31, which receives the fuel control command output from the fuel control unit 25 and controls the supply of fuel gas to the pilot nozzle 16 and main nozzles 17, is provided to control the fuel ratio based on the concentration of inert gas in the fuel gas. Here, the control device 23 and the fuel ratio control unit 31 are electrically connected, and the fuel control command is output to the fuel ratio control unit 31 as an electric signal.

This embodiment describes the case where the fuel ratio control unit 31 is provided outside the control device 23 as an example, but if the fuel ratio control unit 31 is provided inside the control device 23, the fuel ratio control unit 31 may be provided inside the control device 23 as a separate unit from the fuel control unit 25, or may be provided independently within the fuel control unit 25. Further, the fuel ratio control unit 31 can be provided not only as an electronic component but also as a program integrated with the control device 23 or the fuel control unit 25. When the fuel ratio control unit 31 is provided as a program integrated with the control device 23 or the fuel control unit 25, the number of components of the control device 23 can be reduced, and the overall configuration of the control device 23 can be prevented from becoming complex. On the other hand, when the fuel ratio control unit 31 is provided independently as an electronic component, in contrast to when it is provided as a program integrated with the other device, it is possible to prevent multiple control units from failing simultaneously and improve workability because each component can be repaired or updated independently in the event of failure or updating the control contents.

### Operation of Composition Analysis Device for Fuel Gas According to Embodiment of Present Disclosure

Next, the operation of the composition analysis device for fuel gas (composition analysis method for fuel gas) according to an embodiment of the present disclosure will be described. As shown in FIG. 1, when fuel gas supplied to the combustor 4 flows through the fuel supply line 6, the density measurement device 21 and the heating value measurement device 22 measure the density ρ₀ of the fuel gas and the heating value LHV₀ per unit amount of the fuel gas, respectively. Data on the measured density ρ₀ and heating value LHV₀ are transmitted to the composition calculation unit 24 of the control device 23, as shown in FIG. 3. The operation of the composition calculation unit 24 to calculate the composition of the fuel gas (concentration C of inert gas in the fuel gas) using the measured density ρ₀ and heating value LHV₀ will now be described in detail.

In order for the composition calculation unit 24 to calculate the composition of the fuel gas, in addition to the density ρ₀ and the heating value LHV₀, the density ρ1 of flammable gas contained in the fuel gas, the heating value LHV₁ per unit amount of the flammable gas, and the density ρ₂ of inert gas contained in the fuel gas are required. The flammable gas includes, in addition to methane which is the principal component, ethane, propane, etc., and the density ρ1 varies depending on the composition of the flammable gas. If the composition of the flammable gas changes, the heating value LHV₁ also naturally changes. Therefore, the relationship between the density ρ1 and the heating value LHV₁ of the flammable gas is determined in advance by experiment or calculation, and previously stored in the composition calculation unit 24. The density ρ₂ of the inert gas also varies depending on the composition of the inert gas, but since its composition is usually known, the density ρ₂ based on that composition is previously stored in the composition calculation unit 24. Although the density of gas varies with the temperature and pressure of the gas, the effects of temperature and pressure can be ignored if they can be assumed to remain constant without significant changes during operation of the gas turbine 1. On the other hand, if changes in temperature and pressure during operation of the gas turbine 1 cannot be ignored, the effect of temperature and pressure may be included in the relationship between the density ρ1 and the heating value LHV₁ of the flammable gas, and the density ρ₂ of the inert gas may be a function of temperature and pressure. The following explanation is based on the condition that there is no significant change in temperature or pressure of the gas.

When the unit of the concentration C of the inert gas in the fuel gas is the mole fraction, the relationship between the measured density ρ₀ of the fuel gas, the density ρ1 of the flammable gas contained in the fuel gas, and the density ρ₂ of the inert gas contained in the fuel gas is expressed by Eq. (1):

ρ₂C+ρ₁(1−C)=ρ0 (1)

Further, since inert gas does not burn and therefore has zero heating value, the relationship between the measured heating value LHV₀ of the fuel gas and the heating value LHV₁ of the flammable gas is expressed by Eq. (2):

0*C+LHV₁(1−C)=LHV₀ (2)

From Eq. (2), we obtain Eq. (3):

C=1−LHV₀/LHV₁ (3)

By substituting Eq. (3) into Eq. (1), we obtain Eq. (4):

$$\begin{matrix}( Expression 1 ) & \\ {ρ}_{2} ( 1 - \frac{{LHV}_{0}}{{LHV}_{1}} ) + {ρ}_{2} \frac{{LHV}_{0}}{{LHV}_{1}} = ρ & ( 4 )\end{matrix}$$

Here, assume that the relationship between the density ρ₁ and the heating value LHV₁ of the flammable gas stored in the composition calculation unit 24 is a linear regression function as in Eq. (5):

LHV₁=αρ₁+β (5)

In Eq. (5), α and β are constants.

By obtaining LHV₀/LHV₁ from Eqs. (4) and (5) and substituting it into Eq. (3), the following Eq. (6) for the concentration C of the inert gas in the fuel gas can be obtained.

$$\begin{matrix}( Expression 2 ) & \\ C = 1 + \frac{{LHV}_{0}}{α \frac{( {ρ}_{2} - {ρ}_{0} ) β - {ρ}_{0} {LHV}_{0}}{( {ρ}_{2} - {ρ}_{0} ) α + {LHV}_{0}} - β} & ( 6 )\end{matrix}$$

The composition calculation unit 24 calculates the concentration C of the inert gas in the fuel gas, i.e., the composition of the fuel gas based on Eq. (6), from the density ρ₀ and the heating value LHV₀ measured by the density measurement device 21 and the heating value measurement device 22, respectively, the density ρ₂ of the inert gas stored in the composition calculation unit 24, and the function expressed by Eq. (5).

Thus, the heating value LHV₀ per unit amount of the fuel gas and the density ρ₀ of the fuel gas, which can be quickly measured, are measured, and these measured values are used to analyze the composition of the fuel gas, so it is possible to analyze the composition of fuel gas including inert gas and flammable gas quickly.

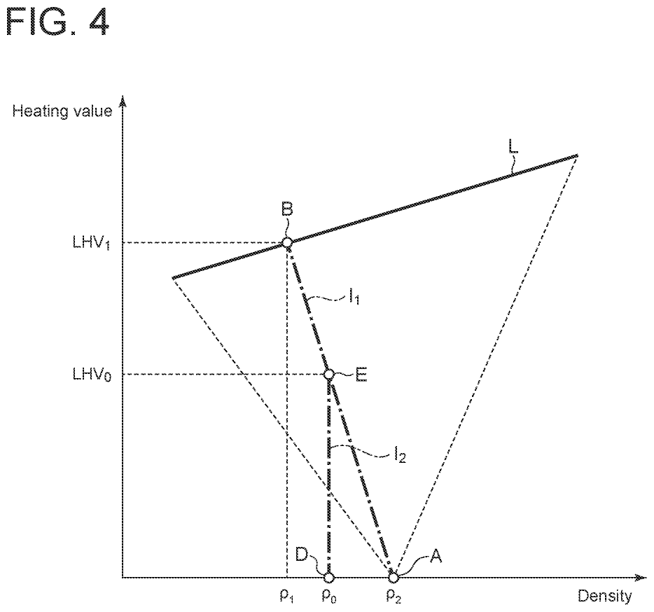

As shown in FIG. 4, on the xy plane where the x-axis is the density and the y-axis is the heating value, the function expressed by Eq. (5) is the straight line L drawn by the solid line. Since inert gas does not burn and therefore has zero heating value, ρ₂ of the inert gas is at point A on the x-axis. On the other hand, the density ρ₁ and the heating value LHV₁ of the flammable gas contained in the combustion gas are represented by point B on the straight line L. Let E be the intersection between the straight line l₁ drawn by the dotted and dashed line connecting points A and B and the straight line 12 drawn by the dotted and dashed line parallel to the y-axis and passing through point D, which represents the measured value of density of the fuel gas on the x-axis, the y-coordinate of the intersection E represents the heating value LHV₀. On this xy plane, the concentration C of the inert gas in the fuel gas, whose unit is the mole fraction, corresponds to the ratio of the length between points B and E to the length between points A and B.

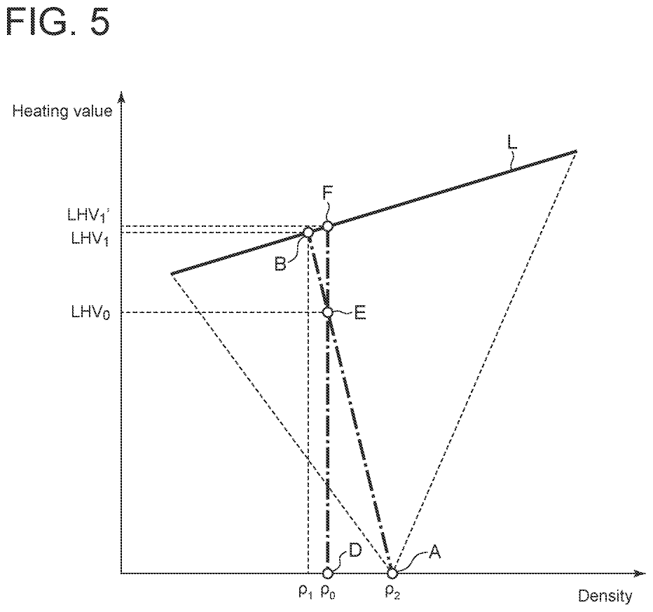

When the concentration of the inert gas in the fuel gas is low, for example several percent or less, the concentration C can be calculated approximately from a simpler equation than Eq. (6). When the concentration of the inert gas in the fuel gas is low, as shown in FIG. 5, points B and F are very close to each other, where F is a point on the straight line L corresponding to the density ρ₀ measured by the density measurement device 21. Therefore, if the heating value corresponding to point F is LHV₁′, the heating value LHV₁′ is approximately equal to the heating value LHV₁ corresponding to point B.

Here,

LHV₁′=αρ₀+β (7)

So, if we use LHV₁′ in Eq. (7) instead of LHV₁ in Eq. (3), Eq. (3) is rewritten as Eq. (8):

$$\begin{matrix}( Expression 3 ) & \\ C = 1 - \frac{{LHV}_{0}}{{LHV}_{1}^{′}} = 1 - \frac{{LHV}_{0}}{{αρ}_{0} + β} & ( 8 )\end{matrix}$$

In this case, the relationship between the density ρ₁ and the heating value LHV₁ of the flammable gas is not limited to a linear regression function as in Eq. (5), but may be any function LHV₁=f(ρ₁). Then, Eq. (7) is: LHV₁′=f(ρ₀) (7′), so Eq. (8) is rewritten as Eq. (9):

$$\begin{matrix}( Expression 4 ) & \\ C = 1 - \frac{{LHV}_{0}}{f ( {ρ}_{0} )} & ( 9 )\end{matrix}$$

Thus, when the concentration of the inert gas in the fuel gas is low, the concentration C can be calculated approximately from the relatively simple Eq. (8) or (9), so it is possible to analyze the composition of fuel gas including inert gas and flammable gas simply.

<Operation of Prime Mover Control Device According to Embodiment of Present Disclosure>

Next, the operation of the prime mover control device according to an embodiment of the present disclosure will be described. As shown in FIG. 3, the fuel control unit 25 receives data on the concentration C of the inert gas in the fuel gas and the heating value LHV₀ per unit amount of the fuel gas from the composition calculation unit 24, and calculates the fuel ratio, which is the ratio of the fuel gas supplied to each of the pilot nozzle 16 (see FIG. 2) and the main nozzles 17 (see FIG. 2), based on the concentration C of the inert gas in the fuel gas.

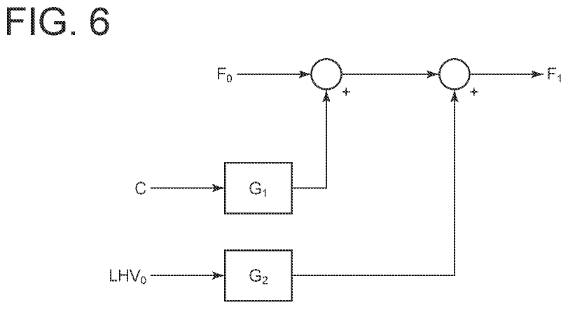

FIG. 6 shows an example of the control flow for the fuel control unit 25 to calculate the fuel ratio from the concentration C and the heating value LHV₀. While the standard fuel ratio F₀ is determined according to the output of the gas turbine 1 in normal operation, the fuel control unit 25 determines fuel ratio gains G₁ and G₂ based on the concentration C and the heating value LHV₀, respectively, and adds them to the standard fuel ratio F₀ to calculate a fuel ratio F₁ based on the concentration C of the inert gas in the fuel gas.

As shown in FIG. 3, the fuel control unit 25 calculates a fuel control command for controlling the fuel ratio control unit 31 so as to achieve the calculated fuel ratio, and outputs the fuel control command to the fuel ratio control unit 31. As a result, the fuel ratio control unit 31 is controlled to supply fuel to the pilot nozzle 16 and the main nozzles 17 at a fuel ratio based on the concentration C of the inert gas in the fuel gas. Thus, even if the concentration C of the inert gas changes, the fuel gas can be stably burned.

In an embodiment of the present disclosure, in order to calculate the concentration C of the inert gas in the fuel gas, the measured values of the density ρ₀ and the heating value LHV₀ by the density measurement device 21 and the heating value measurement device 22 are continuously acquired by the composition calculation unit 24 of the control device 23, and the concentration C of the inert gas in the fuel gas is obtained and databased as appropriate based on the continuously acquired values. However, the control device 23 may be pre-programmed to obtain the concentration C of the inert gas in the fuel gas as data by a series of processes every predetermined period set in advance.

The contents described in the above embodiments would be understood as follows, for instance.

[1] A composition analysis device for fuel gas according to one aspect is a composition analysis device (20) for fuel gas containing inert gas and flammable gas, comprising: a heating value measurement device (22) for measuring a heating value per unit amount of the fuel gas; a density measurement device (21) for measuring a density of the fuel gas; and a control device (23) including a composition calculation unit (24) for calculating a composition of the fuel gas using the heating value measured by the heating value measurement device (22) and the density measured by the density measurement device (21).

With the composition analysis device for fuel gas according to the present disclosure, the heating value per unit amount of the fuel gas and the density of the fuel gas, which can be quickly measured, are measured, and these measured values are used to analyze the composition of the fuel gas, so it is possible to analyze the composition of fuel gas including inert gas and flammable gas quickly.

[2] A composition analysis device for fuel gas according to another aspect is the composition analysis device for fuel gas as defined in [1], where a function representing a relationship of a heating value LHV₁ per unit amount of the flammable gas with respect to a density ρ₁ of the flammable gas is previously defined in the control device (23), and the composition calculation unit (24) is configured to calculate the composition of the fuel gas using the heating value measured by the heating value measurement device (22), the density measured by the density measurement device (21), and the function.

With this configuration, the heating value per unit amount of the fuel gas and the density of the fuel gas, which can be quickly measured, are measured, and these measured values are used to analyze the composition of the fuel gas, so it is possible to analyze the composition of fuel gas including inert gas and flammable gas quickly. Additionally, since the function representing a relationship of the heating value per unit amount of the flammable gas with respect to the density of the flammable gas is previously defined, even if the concentration of the inert gas in the fuel gas changes from moment to moment, it is possible to grasp the concentration quickly.

[3] A composition analysis device for fuel gas according to still another aspect is the composition analysis device for fuel gas as defined in [2], where given that the function is LHV₁=αρ₁+β where α and β are constants, the composition calculation unit (24) calculates a concentration C of the inert gas in the fuel as the composition of the fuel gas, based on the following equation:

$$C = 1 + \frac{{LHV}_{0}}{α \frac{( {ρ}_{2} - {ρ}_{0} ) β - {ρ}_{0} {LHV}_{0}}{( {ρ}_{2} - {ρ}_{0} ) α + {LHV}_{0}} - β} \qquad ( Expression 5 )$$

where LHV₀ is the heating value measured by the heating value measurement device (22), ρ₀ is the density measured by the density measurement device (21), ρ₂ is the density of the inert gas, C is the concentration of the inert gas in the fuel gas.

With this configuration, using the heating value per unit amount of the fuel gas and the density of the fuel gas measured, the concentration C of the inert gas is analyzed as the composition of the fuel gas based on the above equation, so it is possible to analyze the composition of fuel gas including inert gas and flammable gas accurately.

[4] A composition analysis device for fuel gas according to still another aspect is the composition analysis device for fuel gas as defined in [2], where given that the function is LHV₁=f(ρ₁), the composition calculation unit (24) calculates a concentration C of the inert gas in the fuel as the composition of the fuel gas, based on the following equation using f(ρ₀) obtained by substituting ρ₀ for the variable ρ₁ of the function:

$$C = 1 - \frac{{LHV}_{0}}{f ( {ρ}_{0} )} \qquad ( Expression 6 )$$

where LHV₀ is the heating value measured by the heating value measurement device (22), ρ₀ is the density measured by the density measurement device (21), ρ₂ is the concentration of the inert gas in the fuel gas.

With this configuration, when the concentration C of the inert gas in the fuel gas is low, for example several percent or less, the concentration C can be calculated approximately from the relatively simple equation, so it is possible to analyze the composition of fuel gas including inert gas and flammable gas simply.

[5] A composition analysis device for fuel gas according to still another aspect is the composition analysis device for fuel gas as defined in [4], where given that the function is f(ρ₁)=αρ₁+β where α and β are constants, the composition calculation unit (24) calculates the concentration C of the inert gas in the fuel as the composition of the fuel gas, based on the following equation using (αρ₀+β) obtained by substituting ρ₀ for the variable ρ₁ of the function:

$$C = 1 - \frac{{LHV}_{0}}{f ( {ρ}_{0} )} \qquad ( Expression 7 )$$

With this configuration, when the concentration C of the inert gas in the fuel gas is low, for example several percent or less, the concentration C can be calculated approximately from the simpler equation than the equation of the above [4], so it is possible to analyze the composition of fuel gas including inert gas and flammable gas more simply.

[6] A prime mover control device according to one aspect is a prime mover control device (30) for controlling a prime mover (gas turbine 1) provided with a combustor (4) for burning the fuel gas, comprising: the composition analysis device (20) defined in any one of [1] to [5]; and a fuel ratio control unit (31) for adjusting a fuel ratio which is a ratio of the fuel gas supplied to each of different first nozzle (pilot nozzle 16) and second nozzle (main nozzles 17) of the combustor (4). The control device (23) further includes a fuel control unit (25), and the fuel control unit (25) is configured to calculate a fuel control command for correcting the fuel ratio, which is the ratio of the fuel gas, based on the composition of the fuel gas obtained by the composition analysis device (20), and output the fuel control command to the fuel ratio control unit (31).

With the prime mover control device according to the present disclosure, since the fuel ratio, which is the ratio of the fuel gas supplied to the different first and second nozzles of the combustor, is controlled based on quick analysis result of the composition of fuel gas including inert gas and flammable gas, it is possible to maintain proper combustion characteristics in the combustor.

[7] A prime mover control device according to another aspect is the prime mover control device as defined in [6], where the fuel ratio control unit (31) is disposed inside the control device (23) and configured to control the fuel ratio by a program in response to the fuel control command.

With this configuration, since the fuel ratio control unit is provided inside the control device, the number of components of the prime mover control device can be reduced.

[8] A composition analysis method for fuel gas according to one aspect is a composition analysis method for fuel gas containing inert gas and flammable gas, comprising: a step of measuring a heating value per unit amount of the fuel gas; a step of measuring a density of the fuel gas; and a step of calculating a composition of the fuel gas using the heating value and the density measured.

With the composition analysis method for fuel gas according to the present disclosure, the heating value per unit amount of the fuel gas and the density of the fuel gas, which can be quickly measured, are measured, and these measured values are used to analyze the composition of the fuel gas, so it is possible to analyze the composition of fuel gas including inert gas and flammable gas quickly.

[9] A composition analysis method for fuel gas according to another aspect is the composition analysis method for fuel gas as defined in [8], where a function representing a relationship of a heating value LHV₁ per unit amount of the flammable gas with respect to a density ρ₁ of the flammable gas is previously defined, and the composition of the fuel gas is calculated using the heating value and the density measured and the function.

With this method, the heating value per unit amount of the fuel gas and the density of the fuel gas, which can be quickly measured, are measured, and these measured values are used to analyze the composition of the fuel gas, so it is possible to analyze the composition of fuel gas including inert gas and flammable gas quickly.

[10] A composition analysis method for fuel gas according to still another aspect is the composition analysis method for fuel gas as defined in [9], where given that the function is LHV₁=αρ₁+β where α and β are constants, a concentration C of the inert gas in the fuel is calculated as the composition of the fuel gas, based on the following equation:

$$C = 1 + \frac{{LHV}_{0}}{α \frac{( {ρ}_{2} - {ρ}_{0} ) β - {ρ}_{0} {LHV}_{0}}{( {ρ}_{2} - {ρ}_{0} ) α + {LHV}_{0}} - β} \qquad ( Expression 8 )$$

where LHV₀ is the heating value measured, ρ₀ is the density measured, ρ₂ is the density of the inert gas, C is the concentration of the inert gas in the fuel gas.

With this method, using the heating value per unit amount of the fuel gas and the density of the fuel gas measured, the concentration C of the inert gas is analyzed as the composition of the fuel gas based on the above equation, so it is possible to analyze the composition of fuel gas including inert gas and flammable gas accurately.

[11] A composition analysis method for fuel gas according to still another aspect is the composition analysis method for fuel gas as defined in [9], where given that the function is LHV₁=f(ρ₁), a concentration C of the inert gas in the fuel is calculated as the composition of the fuel gas, based on the following equation using f(ρ₀) obtained by substituting ρ₀ for the variable ρ₁ of the function:

$$C = 1 - \frac{{LHV}_{0}}{f ( {ρ}_{0} )} \qquad ( Expression 9 )$$

where LHV₀ is the heating value measured, ρ₀ is the density measured, C is the concentration of the inert gas in the fuel gas.

With this method, when the concentration C of the inert gas in the fuel gas is low, for example several percent or less, the concentration C can be calculated approximately from the relatively simple equation, so it is possible to analyze the composition of fuel gas including inert gas and flammable gas simply.

[12] A composition analysis method for fuel gas according to still another aspect is the composition analysis method for fuel gas as defined in [11], where given that the function is f(ρ₁)=αρ₁+β where α and β are constants, the concentration C of the inert gas in the fuel is calculated as the composition of the fuel gas, based on the following equation using (αρ₀+β) obtained by substituting ρ₀ for the variable ρ₁ of the function:

$$C = 1 - \frac{{LHV}_{0}}{{αρ}_{0} + β} \qquad ( Expression 10 )$$

With this method, when the concentration C of the inert gas in the fuel gas is low, for example several percent or less, the concentration C can be calculated approximately from the simpler equation than the equation of the above [11], so it is possible to analyze the composition of fuel gas including inert gas and flammable gas more simply.

[13] A prime mover control method according to one aspect is a prime mover control method for controlling a prime mover (gas turbine 1) provided with a combustor (4) for burning the fuel gas, comprising: the composition analysis method defined in any one of [8] to [12]; and calculating and outputting a fuel control command for correcting a fuel ratio which is a ratio of the fuel gas supplied to each of different first nozzle (pilot nozzle 16) and second nozzle (main nozzles 17) of the combustor (4), based on the composition of the fuel gas obtained by the composition analysis method.

With the prime mover control method according to the present disclosure, since the fuel ratio, which is the ratio of the fuel gas supplied to the different first and second nozzles of the combustor, is controlled based on quick analysis result of the composition of fuel gas including inert gas and flammable gas, it is possible to maintain proper combustion characteristics in the combustor.

### REFERENCE SIGNS LIST

1 Gas turbine (Prime mover)
4 Combustor
25 16 Pilot nozzle (First nozzle)
17 Main nozzle (Second nozzle)
20 Composition analysis device
21 Density measurement device
22 Heating value measurement device
23 Control device
24 Composition calculation unit
25 Fuel control unit
30 Prime mover control device
31 Fuel ratio control unit

1. A composition analysis device for fuel gas containing inert gas and flammable gas, comprising:
a heating value measurement device for measuring a heating value per unit amount of the fuel gas;
a density measurement device for measuring a density of the fuel gas; and
a control device including a composition calculation unit for calculating a composition of the fuel gas using the heating value measured by the heating value measurement device and the density measured by the density measurement device.

2. The composition analysis device for fuel gas according to claim 1,
wherein a function representing a relationship of a heating value LHV₁ per unit amount of the flammable gas with respect to a density ρ₁ of the flammable gas is previously defined in the control device, and
wherein the composition calculation unit is configured to calculate the composition of the fuel gas using the heating value measured by the heating value measurement device, the density measured by the density measurement device, and the function.

3. The composition analysis device for fuel gas according to claim 2,
wherein, given that the function is LHV₁=αρ₁+β where α and β are constants,
the composition calculation unit calculates a concentration C of the inert gas in the fuel as the composition of the fuel gas, based on the following equation:
$$C = 1 + \frac{{LHV}_{0}}{α \frac{( {ρ}_{2} - {ρ}_{0} ) β - {ρ}_{0} {LHV}_{0}}{( {ρ}_{2} - {ρ}_{0} ) α + {LHV}_{0}} - β}$$
where LHV₀ is the heating value measured by the heating value measurement device,
ρ₀ is the density measured by the density measurement device,
ρ₂ is the density of the inert gas,
C is the concentration of the inert gas in the fuel gas.

4. The composition analysis device for fuel gas according to claim 2,
wherein, given that the function is LHV₁=f(ρ₁),
the composition calculation unit calculates a concentration C of the inert gas in the fuel as the composition of the fuel gas, based on the following equation using f(ρ₀) obtained by substituting ρ₀ for the variable ρ₁ of the function:
$$C = 1 - \frac{{LHV}_{0}}{f ( {ρ}_{0} )}$$
where LHV₀ is the heating value measured by the heating value measurement device,
ρ₀ is the density measured by the density measurement device,
C is the concentration of the inert gas in the fuel gas.

5. The composition analysis device for fuel gas according to claim 4,
wherein, given that the function is f(ρ₁)=αρ₁+β where α and β are constants,
the composition calculation unit calculates the concentration C of the inert gas in the fuel as the composition of the fuel gas, based on the following equation using (αρ₀+β) obtained by substituting ρ₀ for the variable ρ₁ of the function:
$$C = 1 - \frac{{LHV}_{0}}{{αρ}_{0} + β}$$

6. A prime mover control device for controlling a prime mover provided with a combustor for burning the fuel gas, comprising:
the composition analysis device according to claim 1; and
a fuel ratio control unit for adjusting a fuel ratio which is a ratio of the fuel gas supplied to each of different first and second nozzles of the combustor,
wherein the control device further includes a fuel control unit, and
wherein the fuel control unit is configured to calculate a fuel control command for correcting the fuel ratio based on the composition of the fuel gas obtained by the composition analysis device, and output the fuel control command to the fuel ratio control unit.

7. The prime mover control device according to claim 6,
wherein the fuel ratio control unit is disposed inside the control device and configured to control the fuel ratio by a program in response to the fuel control command.

8. A composition analysis method for fuel gas containing inert gas and flammable gas, comprising:
a step of measuring a heating value per unit amount of the fuel gas;
a step of measuring a density of the fuel gas; and
a step of calculating a composition of the fuel gas using the heating value and the density measured.

9. The composition analysis method for fuel gas according to claim 8,
wherein a function representing a relationship of a heating value LHV₁ per unit amount of the flammable gas with respect to a density ρ₁ of the flammable gas is previously defined, and
wherein the composition of the fuel gas is calculated using the heating value and the density measured and the function.

10. The composition analysis method for fuel gas according to claim 9,
wherein, given that the function is LHV₁=αρ₁+β where α and β are constants,
a concentration C of the inert gas in the fuel is calculated as the composition of the fuel gas, based on the following equation:
$$C = 1 + \frac{{LHV}_{0}}{α \frac{( {ρ}_{2} - {ρ}_{0} ) β - {ρ}_{0} {LHV}_{0}}{( {ρ}_{2} - {ρ}_{0} ) α + {LHV}_{0}} - β}$$
where LHV₀ is the heating value measured,
ρ₀ is the density measured,
ρ₂ is the density of the inert gas,
C is the concentration of the inert gas in the fuel gas.

11. The composition analysis method for fuel gas according to claim 9,
wherein, given that the function is LHV₁=f(ρ₁),
a concentration C of the inert gas in the fuel is calculated as the composition of the fuel gas, based on the following equation using f(ρ₀) obtained by substituting ρ₀ for the variable ρ₁ of the function:
$$C = 1 - \frac{{LHV}_{0}}{f ( {ρ}_{0} )}$$
where LHV₀ is the heating value measured,
ρ₀ is the density measured,
C is the concentration of the inert gas in the fuel gas.

12. The composition analysis method for fuel gas according to claim 11,
wherein, given that the function is f(ρ₁)=αρ₁+β where α and β are constants,
the concentration C of the inert gas in the fuel is calculated as the composition of the fuel gas, based on the following equation using (αρ₀+β) obtained by substituting ρ₀ for the variable ρ₁ of the function:
$$C = 1 - \frac{{LHV}_{0}}{{αρ}_{0} + β}$$

13. A prime mover control method for controlling a prime mover provided with a combustor for burning the fuel gas, comprising:
the composition analysis method according to claim 8; and
calculating and outputting a fuel control command for correcting a fuel ratio which is a ratio of the fuel gas supplied to each of different first and second nozzles of the combustor, based on the composition of the fuel gas obtained by the composition analysis method.
